(19) United States
(12) Patent Application Publication
(10) Pub. No.: US 2022/0317133 A1
(43) Pub. Date: Oct. 6, 2022

(54) Assay for Assessing Heart Failure

(71) Applicants: Nordic Bioscience A/S, Herlev (DK); Bristol-Myers Squibb Company, Princeton, NJ (US); The Trustees of the University of Pennsylvania, Philadelphia, PA (US)

(72) Inventors: Federica Genovese, Frederiksberg (DK); Morten Karsdal, København Ø (DK); Lei Zhao, Princeton, NJ (US); David Gordon, Princeton, NJ (US); Zhaoqing Wang, Princeton, NJ (US); Julio Alonso Chirinos Medina, Garnet Valley, PA (US)

(73) Assignees: Nordic Bioscience A/S, Herlev (DK); Bristol-Myers Squibb Company, Princeton, NJ (US); The Trustees of the University of Pennsylvania, Philadelphia, PA (US)

(21) Appl. No.: 17/616,296

(22) PCT Filed: Jun. 5, 2020
(86) PCT No.: PCT/EP2020/065698

§ 371 Date: Dec. 3, 2021

Related U.S. Application Data
(60) Provisional application No. 62/857,362, filed on Jun. 5, 2019.

(51) Int. Cl. G01N 33/68

(52) U.S. Cl. CPC G01N 33/6893; G01N 2800/325; G01N 2333/4716

(57) ABSTRACT

A method of immunoassay for detecting and/or monitoring a cardiovascular disease in a patient and/or assessing the likelihood of or the severity of a cardiovascular disease in a patient, comprising contacting a biofluid sample from a patient with a monoclonal antibody that specifically binds to a C-terminal epitope of the C5 domain of the α3 chain of type VI collagen, and/or contacting a biofluid sample from the patient with a monoclonal antibody that specifically binds to a C-terminal neo-epitope of the N-terminal propeptide of type III collagen.

### FIELD OF THE INVENTION

The present invention relates to immunoassays for detecting and/or monitoring a cardiovascular disease in a patient and/or assessing the likelihood of or the severity of a cardiovascular disease in a patient. The cardiovascular disease may in particular be heart failure, and especially heart failure with preserved ejection fraction. The immunoassay may be for assessing the likelihood of adverse outcomes of the cardiovascular disease. The patient may be a patient undergoing therapy for the cardiovascular disease, such as a patient undergoing treatment with an aldosterone antagonist. The present invention also relates to immunoassays for identifying patients suitable for treatment with an aldosterone antagonist.

### BACKGROUND

The burden of heart failure (HF) has increased dramatically over the last several years^{1,2}. Approximately half of HF is secondary to HF with preserved ejection fraction (HFpEF), which is anticipated to represent an even larger proportion of the total burden of HF as the population ages³. Despite multiple phase-III randomized controlled trials over the last few decades, a pharmacologic intervention proven to provide a clear benefit for this patient population remains to be identified.

The heterogeneity of the HFpEF syndrome has been identified as an important barrier to demonstrating the effectiveness of candidate pharmacologic interventions. Given the heterogenous nature of HFpEF, different degrees of contribution from various pathophysiological processes may unfavorably influence average responses to pharmacologic therapies tested in clinical trials. Therefore, the availability of simple, non-invasive biomarkers capable of readily identifying relevant underlying specific biologic processes that can be targeted with pharmacologic interventions represents a promising approach to enhance our clinical and therapeutic approach to HFpEF⁴.

The heterogeneity of HFpEF also has important implications for the differential prognosis of individual patients. The ability to more effectively risk-stratify HFpEF patients is greatly needed. Novel risk-stratification markers may not only improve our ability to prognosticate HFpEF patients in clinical practice, but would be of great value to inform enrollment of high-risk individuals in future trials.

Myocardial fibrosis is thought to play a role in the pathophysiology of HFpEF^{5,6}. Increased fibrosis results from an excess of formation relative to degradation of collagen, ultimately leading increased interstitial collagen deposition in the interstitium. Increased myocardial extracellular matrix deposition has been demonstrated in HFpEF in autopsy specimens and in vivo studies⁶⁻⁸ and has been shown to correlate with LV passive stiffening and diastolic dysfunction in this condition^{5,8}. Myocardial fibrosis may also contribute to reduced coronary flow reserve^{6,9}, ventricular dyssynchrony and a propensity to arrhythmia^{10,11}. Given the role of myocardial fibrosis in HFpEF, simple fibrotic biomarkers that reflect the underlying dynamic process of fibrosis progression or regression of fibrosis would be highly valuable¹⁰.

The extracellular volume fraction (ECVF), an index of myocardial fibrosis measured by cardiac magnetic resonance imaging, has been reported to predict adverse outcomes in patients with HFpEF^{12,13,} or at risk for HFpEF¹⁴. Although MRI will likely have an important role in the assessment of myocardial fibrosis in preclinical studies, early-phase research in humans and some clinical settings, its cost and availability are likely to limit or preclude its use in global phase-III trials and in clinical practice. In addition, many patients with HFpEF are not candidates for ECVF measurements due to claustrophobia or advanced renal disease. Thus, the search for circulating biomarkers of tissue fibrosis remains an area of great interest.

The search for suitable biomarkers for HFpEF is, however, complicated by the notion that “fibrosis is not just fibrosis”, and that ECM remodeling of different compartments and collagen types may have different biologic and prognostic implications¹⁵. For instance, differential associations of collagen neoepitope fragments and liver fibrosis has been reported in chronic hepatitis B vs. hepatitis C. Moreover, although myocardial fibrosis is thought to be important in HFpEF, extracardiac fibrosis may also play an important role. For example, fibrofatty infiltration of skeletal muscle has been reported in HFpEF¹⁶. Similarly, fibrosis may also occur in the arterial wall, the kidney and the liver dysfunction, all of which may contribute to adverse outcomes in this population.

There is also a need for biomarkers of collagen turnover can identify individuals who benefit from aldosterone antagonists, such as spironolactone. Although this concept has been assessed in previous studies in other populations (HF with reduced ejection fraction and/or post-myocardial infarction)¹⁷, only one study to date has examined this issue in HFpEF¹⁸. In this previous study, the ratio of serum carboxy-terminal telopeptide of collagen type I to serum matrix metalloproteinase-1 (CITP:MMP-1) appeared to identify patients who exhibited reductions in the echocardiographic mitral inflow to annular tissue velocity ratio (a marker of left ventricular filling pressures) after 12 weeks of therapy with spironolactone¹⁸. However, this study did not examine clinical events.

Type III collagen is expressed in most of the type I collagen containing tissues except for bone, and is an important component of connective tissues, muscle tissues and skin. Collagen type III is essential for collagen type I fibrillogenesis in the cardiovascular system and other organs. During fibrillar assembly the N-terminal propeptide of type III procollagen (which consists of three identical α-chains with a total molecular weight of 42 kDa) is cleaved off by specific N-proteases prior to incorporation of the mature collagen in the extracellular matrix (ECM). The cleaved propeptides may either be retained in the ECM or released into the circulation. However, the cleavage of the propeptide is sometimes incomplete, leaving the propeptide attached to the molecule. This results in the formation of thin fibrils with abnormal cross-links, which in turn causes the abnormal molecule to be prone to rapid metabolic turnover. PIIINP is the N-terminal propeptide of collagen type III, which is removed during mature type III collagen synthesis. Thus, the level of the N-terminal propeptide of type III collagen (PIIINP) in a suitable sample can be a marker of formation and/or degradation of collagen type III.

PRO-C3 is a biomarker for formation of collagen type III, comprising a C-terminal neo-epitope of the N-terminal propeptide of type III collagen (i.e. a C-terminal neo-epitope of PIIINP), which neo-epitope is formed after the cleavage of the pro-peptide from the pro-collagen by ADAMTS-2. The PRO-C3 biomarker, and a PRO-C3 assay (specifically, a PRO-C3 ELISA) are described in WO2014/170312. The assay utilizes a monoclonal antibody that specifically binds to the C-terminus 10 amino acid sequence of PIIINP, and so targets the free C-terminal end of the N-terminal pro-peptide that is formed after cleavage¹⁹. PRO-C3 is a well explored biomarker of liver fibrosis, related both to fibrotic burden in the liver and to progression of fibrosis and adverse outcome in patients with different liver indications²⁰⁻²⁶.

Collagen Type VI is a unique extracellular collagen which can form an independent microfibrillar network in the basement membrane of cells. It can interact with other matrix proteins including collagens, biglycan, and proteoglycans. In muscle, type VI collagen is part of the sarcolemma and is involved in anchoring the muscle fiber into the intramuscular extracellular matrix, and so is involved in force transmission. Moreover, mutations in type VI collagen can cause Bethlem myopathy and Ullrich congenital muscular dystrophy. It has been reported that the C-terminal amino acid sequence of the type VI collagen α3 chain is cleaved off from the mature type VI microfibril after secretion. However, Type VI collagen is not just involved in muscles and muscle loss.

The microflamentous interstitial type VI collagen, a triple helical molecule composed of the constituent chains α1(VI), α2(VI), and α3(VI), is expressed in most connective tissues and prominently in adipose tissue, where it anchors cells through its interconnections with other ECM proteins. During the formation of microfilaments, the triple-helical core of type VI collagen is proteolytically released from the pro-peptide, and cleavage of the C-terminal pro-peptide of the α3(VI) chain generates endotrophin, an adipokine.

PRO-C6 is a biomarker for formation of collagen type VI and endotrophin release, comprising a C-terminal epitope of the C5 domain of the α3 chain of type VI collagen that is cleaved off when a novel collagen type VI molecule assembles in the extracellular matrix, and which C-terminal epitope is also a C-terminal epitope of the bioactive fragment endotrophin. The PRO-C6 biomarker, and a PRO-C6 assay (specifically, a PRO-C6 ELISA) are described in WO2016/156526. The assay utilizes a monoclonal antibody that specifically binds to the C-terminus 10 amino acid sequence of the C5 domain of the α3 chain of collagen type VI. Endotrophin's role as a pro-fibrotic, pro-inflammatory and pro-tumorigenic molecule has been observed in preclinical models of breast cancer and liver fibrosis²⁷⁻³¹. PRO-C6 has been established as a prognostic biomarker for mortality and disease progression in chronic kidney disease and diabetic kidney disease patients³²⁻³⁴ and as a predictive marker for response to glucose lowering therapy in diabetic patients³⁵.

Collagen IV is a type of collagen found primarily in the basal lamina of vessels. The vascular wall consists of two major types of extracellular matrix: the basement membrane and the interstitial matrix. The basement membrane is composed of 2 independent polymeric networks: one made of type IV collagen, and made of laminins, in addition to proteoglycans^{36,37} and various other glycoproteins. The collagen IV network in the basement membrane is highly cross-linked and considered to maintain mechanical stability. Basal membranes also harbor matrix metalloproteases (MMPs), a large family of broad-spectrum proteases that function in degradation and remodeling of the basement membrane. By degrading basement membrane scaffolds, MMPs can release cryptic fragments with signaling functions. For example, cleavage of collagen IV by MMP9 exposes a cryptic site involved in angiogenesis³⁷.

C4M is a biomarker for MMP-mediated degradation of type IV collagen, comprising an N-terminal neo-epitope of a fragment of type IV collagen formed by cleavage of the α1(IV) chain by MMP12. The C4M biomarker, and a C4M assay (specifically, a C4M ELISA) have been described previously³⁸. The assay utilizes a monoclonal antibody that specifically binds to said N-terminal neo-epitope.

### SUMMARY

The present inventors have now explored the potential of PRO-C6 and PRO-C3 as biomarkers for cardiovascular diseases. The levels of PRO-C6 and PRO-C3 in the circulation of a cohort of patients with heart failure with preserved ejection fraction (HFpEF) were examined. Baseline PRO-C3 and PRO-C6 levels were analyzed and the relationship between biomarker and outcomes were investigated, and both PRO-C3 and PRO-C6 were found to be effective diagnostic and prognostic biomarkers of heart failure.

In addition, the present inventors have also explored the potential of C4M as a biomarker for patients with cardiovascular disease who may be responsive to treatment with an aldosterone antagonist. The levels of C4M in patients with HFpEF were examined, and C4M was found to identify patients more likely to exhibit a favorable response to treatment with the aldosterone antagonist Spironolactone.

Accordingly, in a first aspect the present invention provides a method of immunoassay for detecting and/or monitoring a cardiovascular disease in a patient and/or assessing the likelihood of or the severity of a cardiovascular disease in a patient, wherein said method comprises:
(i) contacting a biofluid sample from a patient with a monoclonal antibody that specifically binds to a C-terminal epitope of the C5 domain of the α3 chain of type VI collagen, and/or contacting a biofluid sample from the patient with a monoclonal antibody that specifically binds to a C-terminal neo-epitope of the N-terminal propeptide of type III collagen,
(ii) detecting and determining the amount of binding between each monoclonal antibody used in step (i) and peptides in the sample or samples, and
(iii) correlating said amount of binding of each monoclonal antibody as determined in step (ii) with values associated with normal healthy subjects and/or values associated with known disease severity and/or values obtained from said patient at a previous time point and/or a predetermined cut-off value.

The immunoassay may be, but is not limited to, a competition assay or a sandwich assay. The immunoassay may, for example, be a radioimmunoassay or an enzyme-linked immunosorbent assay (ELISA). Such assays are a techniques known to the person skilled in the art.

The cardiovascular disease may in certain embodiments be heart failure. In particular, the cardiovascular disease may be heart failure with a preserved ejection fraction (HFpEF).

The method may in certain embodiments be a method for assessing the severity of a cardiovascular disease in a patient that comprises assessing the likelihood of patient mortality and/or hospitalization as a result of the cardiovascular disease and/or a composite of adverse cardiovascular events.

The patient may, for example, be a patient undergoing a therapy for the cardiovascular disease.

The patient biofluid sample may be, but is not limited to, blood, serum, plasma, urine or amniotic fluid. Preferably the biofluid is serum or plasma.

As used herein the term “monoclonal antibody” refers to both whole antibodies and to fragments thereof that retain the binding specificity of the whole antibody, such as for example a Fab fragment, F(ab′)2 fragment, single chain Fv fragment, or other such fragments known to those skilled in the art. As is well known, whole antibodies typically have a “Y-shaped” structure of two identical pairs of polypeptide chains, each pair made up of one “light” and one “heavy” chain. The N-terminal regions of each light chain and heavy chain contain the variable region, while the C-terminal portions of each of the heavy and light chains make up the constant region. The variable region comprises three complementarity determining regions (CDRs), which are primarily responsible for antigen recognition. The constant region allows the antibody to recruit cells and molecules of the immune system. Antibody fragments retaining binding specificity comprise at least the CDRs and sufficient parts of the rest of the variable region to retain said binding specificity.

In the methods of the present invention, a monoclonal antibody comprising any constant region known in the art can be used. Human constant light chains are classified as kappa and lambda light chains. Heavy constant chains are classified as mu, delta, gamma, alpha, or epsilon, and define the antibody's isotype as IgM, IgD, IgG, IgA, and IgE, respectively. The IgG isotype has several subclasses, including, but not limited to IgGI, IgG2, IgG3, and IgG4. The monoclonal antibody may preferably be of the IgG isotype, including any one of IgGI, IgG2, IgG3 or IgG4.

The CDR of an antibody can be determined using methods known in the art such as that described by Kabat et al. Antibodies can be generated from B cell clones as described in the examples. The isotype of the antibody can be determined by ELISA specific for human IgM, IgG or IgA isotype, or human IgG1, IgG2, IgG3 or IgG4 subclasses. The amino acid sequence of the antibodies generated can be determined using standard techniques. For example, RNA can be isolated from the cells, and used to generate cDNA by reverse transcription. The cDNA is then subjected to PCR using primers which amplify the heavy and light chains of the antibody. For example primers specific for the leader sequence for all VH (variable heavy chain) sequences can be used together with primers that bind to a sequence located in the constant region of the isotype which has been previously determined. The light chain can be amplified using primers which bind to the 3′ end of the Kappa or Lamda chain together with primers which anneal to the V kappa or V lambda leader sequence. The full length heavy and light chains can be generated and sequenced.

In some embodiments of the methods according to the first aspect of the invention, the biofluid sample is contacted with a monoclonal antibody which specifically binds to a C-terminal epitope of the C5 domain of the α3 chain of type VI collagen. Preferably said monoclonal antibody specifically binds to the C-terminus amino acid sequence KPGVISVMGT (SEQ ID No: 1) (also referred to herein as the “PRO-C6 sequence”, or simply “PRO-C6”). Preferably said monoclonal antibody does not recognize or specifically bind to an elongated version of said C-terminus amino acid sequence which is KPGVISVMGTA (SEQ ID No: 2), or to a truncated version of said C-terminus amino acid sequence which is KPGVISVMG (SEQ ID No: 3).

Preferably, the ratio of the affinity of said antibody for the C-terminus amino acid sequence KPGVISVMGT (SEQ ID No: 1) to the affinity of said antibody for the elongated C-terminus amino acid sequence KPGVISVMGTA (SEQ ID No: 2), and/or for the truncated C-terminus amino acid sequence KPGVISVMG (SEQ ID No: 3), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

As used herein the term “C-terminus” refers to a C-terminal peptide sequence at the extremity of a polypeptide, i.e. at the C-terminal end of the polypeptide, and is not to be construed as meaning in the general direction thereof.

The monoclonal antibody that specifically binds to the PRO-C6 sequence may preferably comprises one or more complementarity-determining regions (CDRs) selected from:

CDR-L1:
(SEQ ID No: 4)
RSSQRIVHSNGITFLE
CDR-L2:
(SEQ ID No: 5)
RVSNRFS
CDR-L3:
(SEQ ID No: 6)
FQGSHVPLT
CDR-H1:
(SEQ ID No: 7)
DFNMN
CDR-H2:
(SEQ ID No: 8)
AINPHNGATSYNQKFSG
CDR-H3:
(SEQ ID No: 9)
WGNGKNS.

Preferably the antibody comprises at least 2, 3, 4, 5 or 6 of the above listed CDR sequences.

Preferably the monoclonal antibody light chain variable region comprises the CDR sequences

CDR-L1:
(SEQ ID No: 4)
RSSQRIVHSNGITFLE
CDR-L2:
(SEQ ID No: 5)
RVSNRFS
and
CDR-L3:
(SEQ ID No: 6)
FQGSHVPLT.

Preferably the monoclonal antibody light chain comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID No: 10)
RSSQRIVHSNGITFLEWYLQKPGQSPKLLIYRVSNRFSGVPDRFSG
SGSGTDFTLKISRVEAEDLGLYYCFQGSHVPLT.

Preferably the monoclonal antibody heavy chain variable region comprises the CDR sequences

CDR-H1:
(SEQ ID No: 7)
DFNMN
CDR-H2:
(SEQ ID No: 8)
AINPHNGATSYNQKFSG
and
CDR-H3:
(SEQ ID No: 9)
WGNGKNS.

Preferably the monoclonal antibody heavy chain comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID No: 11)
DFNMNWVKQSHGKSLEWIGAINPHNGATSYNQKFSGKATLTVDKSS
STAYMELNSLTSDDSAVYYCARWGNGKNS.

As used herein, the framework amino acid sequences between the CDRs of an antibody are substantially identical or substantially similar to the framework amino acid sequences between the CDRs of another antibody if they have at least 70%, 80%, 90% or at least 95% similarity or identity. The similar or identical amino acids may be contiguous or non-contiguous.

The framework sequences may contain one or more amino acid substitutions, insertions and/or deletions. Amino acid substitutions may be conservative, by which it is meant the substituted amino acid has similar chemical properties to the original amino acid. A skilled person would understand which amino acids share similar chemical properties. For example, the following groups of amino acids share similar chemical properties such as size, charge and polarity: Group 1 Ala, Ser, Thr, Pro, Gly; Group 2 Asp, Asn, Glu, Gln; Group 3 His, Arg, Lys; Group 4 Met, Leu, Ile, Val, Cys; Group 5 Phe Thy Trp.

A program such as the CLUSTAL program to can be used to compare amino acid sequences. This program compares amino acid sequences and finds the optimal alignment by inserting spaces in either sequence as appropriate. It is possible to calculate amino acid identity or similarity (identity plus conservation of amino acid type) for an optimal alignment. A program like BLASTx will align the longest stretch of similar sequences and assign a value to the fit. It is thus possible to obtain a comparison where several regions of similarity are found, each having a different score. Both types of analysis are contemplated in the present invention. Identity or similarity is preferably calculated over the entire length of the framework sequences.

In certain preferred embodiments, the monoclonal antibody that specifically binds to the PRO-C6 sequence may comprise the light chain variable region sequence:

(SEQ ID No: 12)
DVVMTQTPLSLPVNLGDQASISCRSSQRIVHSNGITFLEWYLQKPG
QSPKLLIYRVSNRFSGVPDRFSGSGSGTDFTLKISRVEAEDLGLYY
CFQGSHVPLTFGAGTRLELK

and/or the heavy chain variable region sequence:

(SEQ ID No: 13)
EVQLQQSGPVMVKPGTSVKTSCKASGYTFTDFNMNWVKQSHGKSLE
WIGAINPHNGATSYNQKFSGKATLTVDKSSSTAYMELNSLTSDDSA
VYYCARWGNGKNSWGQGTTLTVSS

(CDRs bold and underlined; Framework sequences in italics)

In some embodiments of the methods according to the first aspect of the invention, the biofluid sample is contacted with a monoclonal antibody which specifically binds to a C-terminal neo-epitope of the N-terminal propeptide of type III collagen. Preferably, said monoclonal antibody specifically binds to a C-terminus amino acid sequence CPTGPQNYSP (SEQ ID No: 14) (also referred to herein as the “PRO-C3 sequence”, or simply “PRO-C3”). More preferably the monoclonal antibody does not recognize or specifically bind to an elongated version of said C-terminus amino acid sequence which is CPTGPQNYSPQ (SEQ ID No: 15), or to a truncated version of said C-terminus amino acid sequence which is CPTGPQNYS (SEQ ID No: 16).

Preferably, the ratio of the affinity of said antibody for the C-terminus amino acid sequence CPTGPQNYSP (SEQ ID No: 14) to the affinity of said antibody for the elongated C-terminus amino acid sequence CPTGPQNYSPQ (SEQ ID No: 15), and/or for the truncated C-terminus amino acid sequence CPTGPQNYS (SEQ ID No: 16), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

The monoclonal antibody that specifically binds to the PRO-C3 sequence may preferably comprises one or more complementarity-determining regions (CDRs) selected from:

CDR-L1:
(SEQ ID No: 17)
RSSQNIVYSNGDTYFE
CDR-L2:
(SEQ ID No: 18)
KVSQRFS
CDR-L3:
(SEQ ID No: 19)
FQGAHDPPA
CDR-H1:
(SEQ ID No: 20)
GYTFINYVIH
CDR-H2:
(SEQ ID No: 21)
YMNPYNDVPKNNAKFRG
CDR-H3:
(SEQ ID No: 22)
GGFFGPLSY.

Preferably the antibody comprises at least 2, 3, 4, 5 or 6 of the above listed CDR sequences.

Preferably the monoclonal antibody light chain variable region comprises the CDR sequences

CDR-L1:
(SEQ ID No: 17)
RSSQNIVYSNGDTYFE
CDR-L2:
(SEQ ID No: 18)
KVSQRFS
and
CDR-L3:
(SEQ ID No: 19)
FQGAHDPPA.

Preferably the monoclonal antibody light chain comprises framework sequences between the CDRs, wherein said framework sequences are substantially identical or substantially similar to the framework sequences between the CDRs in the light chain sequence below (in which the CDRs are shown in bold and underlined, and the framework sequences are shown in italics)

(SEQ ID No: 23)
RSSQNIVYSNGDTYFEWYLQKPGQSPKLLIYKVSQRFSGVPDRFSG
SGSGTDFTLKISRVETEDLGVYYCFQGAHDPPA.

Preferably the monoclonal antibody heavy chain variable region comprises the CDR sequences

CDR-H1:
(SEQ ID No: 20)
GYTFINYVIH
CDR-H2:
(SEQ ID No: 21)
YMNPYNDVPKNNAKFRG
and
CDR-H3:
(SEQ ID No: 22)
GGFFGPLSY.

Preferably the monoclonal antibody heavy chain comprises framework sequences between the CDRs, wherein said framework sequences are substantially identical or substantially similar to the framework sequences between the CDRs in the heavy chain sequence below (in which the CDRs are shown in bold and underlined, and the framework sequences are shown in italics)

(SEQ ID No: 24)
GYTFINYVIHWLKQKAGQGPEWIGYMNPYNDVPKNNAKFRGKARLT
SDRSSTTAYMELNSLTSEDSAVYYCARGGFFGPLSY.

In certain preferred embodiments, the monoclonal antibody that specifically binds to the PRO-C3 sequence may comprise the light chain variable region sequence:

(SEQ ID No: 25)
DVLMTQTPLSLSVSLGDQASISCRSSQNIVYSNGDTYFEWYLQKPG
QSPKLLIYKVSQRFSGVPDRFSGSGSGTDFTLKISRVETEDLGVYY
CFQGAHDPPAFGGGTKLELK

and/or the heavy chain variable region sequence:

(SEQ ID No: 26)
EVQLQQSGPEVLKPGASVKMSCKASGYTFINYVIHWLKQKAGQGPE
WIGYMNPYNDVPKNNAKFRGKARLTSDRSSTTAYMELNSLTSEDSA
VYYCARGGFFGPLSYWGQGTLVTVSA

(CDRs bold and underlined; Framework sequences in italics)

In some embodiments of the methods according to the first aspect of the invention, the amount of binding of the monoclonal antibody specific for the C-terminal epitope of the C5 domain of the α3 chain of collagen type VI, and/or the amount of binding of the monoclonal antibody specific for the C-terminal neo-epitope of the N-terminal propeptide of type III collagen (PIIINP), are correlated with values associated with normal healthy subjects and/or with values associated with known disease severity and/or with values obtained from the patient at a previous point in time.

As used herein the term “values associated with normal healthy subjects and/or values associated with known disease severity” means standardised quantities determined by the method described supra for subjects considered to be healthy, i.e. without a cardiovascular disease, and/or standardised quantities determined by the method described supra for subjects known to have a cardiovascular disease with a known severity.

In some embodiments of the method according to the first aspect, the amount of binding of the monoclonal antibody specific for the C-terminal epitope of the C5 domain of the α3 chain of collagen type VI, and/or the amount of binding of the monoclonal antibody specific for the C-terminal neo-epitope of N-terminal propeptide of type III collagen (PIIINP), are compared with one or more predetermined cut-off values.

As used herein the “cut-off value” means an amount of binding that is determined statistically to be indicative of a high likelihood of cardiovascular disease in a patient, or of cardiovascular disease of a particular level of severity, in that a measured value of biomarker binding in a patient sample that is at or above the statistical cutoff value corresponds to at least a 70% probability, preferably at least an 80% probability, preferably at least an 85% probability, more preferably at least a 90% probability, and most preferably at least a 95% probability of the presence or likelihood of cardiovascular disease or of a particular level of severity of the disease.

The predetermined cut-off value for the amount of binding of the monoclonal antibody specific for the C-terminal epitope of the C5 domain of the α3 chain of collagen type VI is preferably at least 11.0 ng/mL, more preferably at least 16.0 ng/mL. The predetermined cut-off value for amount of binding of the monoclonal antibody specific for the C-terminal neo-epitope of PIIINP is preferably at least 10.0 ng/mL, more preferably at least 14.0 ng/mL. In this regard, through the combined use of various statistical analyses it has been found that a measured amount of binding of the monoclonal antibody specific for the C-terminal epitope of the C5 domain of the α3 chain of collagen type VI of at least 11 ng/mL or greater, and in particular at least 16.0 ng/mL or greater, may be determinative of cardiovascular disease and/or increased risk of hospitalisation or mortality. By having a statistical cut-off value of at least 11.0 ng/mL, and more preferably at least 16.0 ng/mL it is possible to utilise the method of the invention to give a prognosis of cardiovascular disease and/or increased risk of hospitalisation or mortality with a high level of confidence. Likewise, it has been found that a measured amount of binding of the monoclonal antibody specific for C-terminal neo-epitope of PIIINP of at least 10 ng/mL or greater, and in particular at least 14.0 ng/mL or greater, may be determinative of cardiovascular disease and/or increased risk of hospitalisation or mortality, and by having a statistical cut-off value of at least 10.0 ng/mL PRO-C3, and more preferably at least 14.0 ng/mL it is possible to utilise the method of the invention to give a prognosis of cardiovascular disease and/or increased risk of hospitalisation or mortality with a high level of confidence. Applying such statistical cut-off values are particularly advantageous as it results in a standalone diagnostic assay; i.e. it removes the need for any direct comparisons with healthy individuals and/or patients with known disease severity in order to arrive at a diagnostic conclusion. This may also be particularly advantageous when utilising the assay to evaluate patients that already have medical signs or symptoms that are generally indicative of cardiovascular disease (e.g. as determined by a physical examination and/or consultation with a medical professional) as it may act as a quick and definitive tool for corroborating the initial prognosis and thus potentially remove the need for more invasive procedures, and expedite the commencement of a suitable treatment regimen. It may also avoid the need for a lengthy hospital stay. In the particular case of cardiovascular disease, an expedited conclusive diagnosis may result in the disease being detected at an earlier stage, which may in turn improve overall chances of survival, and/or reduce the risk of hospitalisation.

In a second aspect, the present invention provides a method for monitoring a cardiovascular disease and/or assessing the severity of a cardiovascular disease in a patient undergoing treatment with an aldosterone antagonist, wherein said method comprises:
(i) contacting a biofluid sample from a patient undergoing treatment with an aldosterone antagonist with a monoclonal antibody that specifically binds to a C-terminal epitope of the C5 domain of the α3 chain of type VI collagen, and/or contacting a biofluid sample from the patient with a monoclonal antibody that specifically binds to a C-terminal neo-epitope of the N-terminal propeptide of type III collagen,
(ii) detecting and determining the amount of binding between each monoclonal antibody used in step (i) and peptides in the sample or samples, and
(iii) correlating said amount of binding of each monoclonal antibody as determined in step (ii) with values associated with normal healthy subjects and/or values associated with known disease severity and/or values obtained from said patient at a previous time point and/or a predetermined cut-off value.

Such methods may enable identification and monitoring of patients who respond optimally to the treatment with the aldosterone antagonist. Aldosterone antagonists (also known as antimineralocorticoids) include Spironolactone, Eplerenone, Canrenone, Finerenone and Mexrenone. Preferably the aldosterone antagonist is Spironolactone.

Preferred embodiments of the second aspect of the present invention are as described above in relation to the first aspect.

In a third aspect, the present invention provides a method for identifying a patient, with a cardiovascular disease, who is more likely to respond favourably to treatment with an aldosterone antagonist, wherein said method comprises:
(i) contacting a biofluid sample from a patient with a monoclonal antibody that specifically binds to the N-terminus amino acid sequence ILGHVPGMLL (SEQ ID No: 27) (also referred to herein as the “C4M sequence”, or simply “C4M”), (ii) detecting and determining the amount of binding between the monoclonal antibody
used in step (i) and peptides in the sample or samples, and
iii) correlating said amount of binding of the monoclonal antibody as determined in step (ii) with values associated with patients likely to respond favourably to treatment with an aldosterone antagonist and/or with values associated with patients unlikely to respond favourably to treatment with an aldosterone antagonist and/or with a predetermined cut-off value.

Preferably the aldosterone antagonist is Spironolactone.

The immunoassay may be, but is not limited to, a competition assay or a sandwich assay. The immunoassay may, for example, be a radioimmunoassay or an enzyme-linked immunosorbent assay (ELISA).

The cardiovascular disease may in certain embodiments be heart failure. In particular, the cardiovascular disease may be heart failure with a preserved ejection fraction (HFpEF).

The patient biofluid sample may be, but is not limited to, blood, serum, plasma, urine or amniotic fluid. Preferably the biofluid is serum or plasma.

Preferably the monoclonal antibody does not recognize or specifically bind to an elongated version of said N-terminus amino acid sequence which is EILGHVPGMLL (SEQ ID No: 28), or to a truncated version of said N-terminus amino acid sequence which is LGHVPGMLL (SEQ ID No: 29).

Preferably, the ratio of the affinity of said antibody for the N-terminus amino acid sequence ILGHVPGMLL (SEQ ID No: 27) to the affinity of said antibody for the elongated N-terminus amino acid sequence EILGHVPGMLL (SEQ ID No: 28), and/or for the truncated N-terminus amino acid sequence LGHVPGMLL (SEQ ID No: 29), is at least 10 to 1, and more preferably is at least 50 to 1, at least 100 to 1, at least 500 to 1, at least 1,000 to 1, at least 10,000 to 1, at least 100,000 to 1, or at least 1,000,000 to 1.

As used herein the term “N-terminus” refers to a N-terminal peptide sequence at the extremity of a polypeptide, i.e. at the N-terminal end of the polypeptide, and is not to be construed as meaning in the general direction thereof.

### FIGURES

FIG. 1A: Hazard ratio for the primary endpoint per standard-deviation change in fibrosis biomarkers in unadjusted analyses (one model per biomarker).

FIG. 1B: Hazard ratio for the composite endpoint of death or heart failure admission per standard-deviation change in fibrosis biomarkers in unadjusted analyses (one model per biomarker).

FIG. 2: Kaplan-Meier survival curves for the primary endpoint among subjects stratified by tertiles of Pro-C6 (left) and Pro-C3 (right).

FIG. 3: Kaplan-Meier survival curves for the composite endpoint of death or heart failure admission among subjects stratified by tertiles of Pro-C6 (left) and Pro-C3 (right).

### EXAMPLES

### Example 1—Antibody Development for Pro-C6

A monoclonal antibody specific for Pro-C6 was developed as described in WO 2016/156526 (Nordic Bioscience, incorporated herein by reference) using the last 10 amino acids of the type VI collagen α3 chain (i.e. the C-terminus sequence ³¹⁶⁸‘KPGVISVMGT’³¹⁷⁷(SEQ ID No: 1)) as an immunogenic peptide. Briefly, 4-6-week-old Balb/C mice were immunized subcutaneously with 200 μl emulsified antigen with 60 μg of the immunogenic peptide. Consecutive immunizations were performed at 2-week intervals in Freund's incomplete adjuvant, until stable sera titer levels were reached, and the mice were bled from the 2nd immunization on. At each bleeding, the serum titer was detected and the mouse with highest antiserum titer and the best native reactivity was selected for fusion. The selected mouse was rested for 1 month followed by intravenous boosting with 50 μg of immunogenic peptide in 100 μl 0.9% sodium chloride solution 3 days before isolation of the spleen for cell fusion.

Mouse spleen cells were fused with SP2/0 myeloma fusion partner cells. The fusion cells were raised in 96-well plates and incubated in the CO2-incubator. Here standard limited dilution was used to promote monoclonal growth. Cell lines specific to the selection peptide and without cross-reactivity to either elongated peptide (KPGVISVMGTA (SEQ ID No: 2), Chinese Peptide Company, China) or truncated peptide (KPGVISVMG (SEQ ID No: 3), American Peptide Company, USA) were selected and sub-cloned. At last the antibodies were purified using an IgG column.

The antibodies generated were sequenced and the CDRs determined.

The sequence of the chains are as follows (CDRs underlined and in bold):

Heavy Chain Sequence (mouse IgG1 isotype)
(SEQ ID No: 30)
EVQLQQSGPVMVKPGTSVKTSCKASGYTFTDFNMNWVKQSHGKSLE
WIGAINPHNGATSYNQKFSGKATLTVDKSSSTAYMELNSLTSDDSA
VYYCARWGNGKNSWGQGTTLTVSSAKTTPPSVYPLAPGSAAQTNSM
VTLGCLVKGYFPEPVTVTWNSGSLSSGVHTFPAVLQSDLYTLSSSV
TVPSSTWPSETVTCNVAHPASSTKVDKKIVPRDCGCKPCICTVPEV
SSVFIFPPKPKDVLTITLTPKVTCVVVDISKDDPEVQFSWFVDDVE
VHTAQTQPREEQFNSTFRSVSELPIMHQDWLNGKEFKCRVNSAAFP
APIEKTISKTKGRPKAPQVYTIPPPKEQMAKDKVSLTCMITDFFPE
DITVEWQWNGQPAENYKNTQPIMDTDGSYFVYSKLNVQKSNWEAGN
TFTCSVLHEGLHNHHTEKSLSHSPGK
CDR-H1:
(SEQ ID No: 7)
DFNMN
CDR-H2:
(SEQ ID No: 8)
AINPHNGATSYNQKFSG
CDR-H3:
(SEQ ID No: 9)
WGNGKNS
Light Chain Sequence (mouse Kappa isotype)
(SEQ ID No: 31)
DVVMTQTPLSLPVNLGDQASISCRSSQRIVHSNGITFLEWYLQKPG
QSPKLLIYRVSNRFSGVPDRFSGSGSGTDFTLKISRVEAEDLGLYY
CFQGSHVPLTFGAGTRLELKRADAAPTVSIFPPSSEQLTSGGASVV
CFLNNFYPKDINVKWKIDGSERQNGVLNSWTDQDSKDSTYSMSSTL
TLTKDEYERHNSYTCEATHKTSTSPIVKSFNRNEC
CDR-L1:
(SEQ ID No: 4)
RSSQRIVHSNGITFLE
CDR-L2:
(SEQ ID No: 5)
RVSNRFS
CDR-L3:
(SEQ ID No: 6)
FQGSHVPLT

### Example 2—Antibody Development for Pro-C3

A monoclonal antibody specific for Pro-C3 was developed as described in WO 2014/170312 (Nordic Bioscience, incorporated herein by reference) using sequence 145′-CPTGPQNYSP-′153 (SEQ ID No: 14) of the α1 chain PIIINP as an immunogenic peptide. Briefly, generation of monoclonal antibodies was initiated by subcutaneous immunization of 4-5 week old Balb/C mice with 200 μl emulsified antigen and 50 μg PIIINP neo-epitope C-terminus sequence (OVA-CGG-CPTGPQNYSP (SEQ ID No: 32)) using Freund's incomplete adjuvant. The immunizations were repeated every 2 weeks until stable serum titer levels were reached. The spleen cells were fused with SP2/0 myeloma cells to produce hybridoma, and cloned in culture dishes using the semi-medium method. The supernatants were screened for reactivity against calibrator peptide and native material in an indirect ELISA using streptavidin-coated plates. Biotin-CGG-CPTGPQNYSP (SEQ ID No: 33) was used as screening peptide, while the free peptide CPTGPQNYSP (SEQ ID No: 14) was used as calibrator to test for further specificity of clones.

Native reactivity and affinity of the antibody was assessed using different biological materials such as urine, serum, and amniotic fluid (AF) from both humans and rats in a preliminary ELISA using 2 ng/ml biotinylated peptide on streptavidin-coated microtiter plates and the supernatants from growing monoclonal hybridoma cells. Antibody specificity was tested in a preliminary assay using deselection and elongated peptides (i.e. calibrator peptide with ten amino acid substitutions and calibrator peptide with one additional amino acid at the cleavage site, respectively). The isotype of the monoclonal antibodies was determined using the Clonotyping System-HRP kit, cat. 5300-05 (Southern Biotech, Birmingham, Ala., USA). The subtype was determined to be an IgG2 subtype.

The antibodies generated were sequenced and the CDRs determined.

The sequence of the chains are as follows (CDRs underlined and in bold):

Heavy Chain Sequence (mouse IgG2A isotype)
(SEQ ID No: 34)
EVQLQQSGPEVLKPGASVKMSCKASGYTFINYVIHWLKQKAGQGPEW
IGYMNPYNDVPKNNAKFRGKARLTSDRSSTTAYMELNSLTSEDSAVY
YCARGGFFGPLSYWGQGTLVTVSAAKTTAPSVYPLAPVCGDTTGSSV
TLGCLVKGYFPEPVTLTWNSGSLSSGVHTFPAVLQSDLYTLSSSVTV
TSSTWPSQSITCNVAHPASSTKVDKKIEPRGPTIKP
CDR-H1:
(SEQ ID No: 20)
GYTFINYVIH
CDR-H2:
(SEQ ID No: 21)
YMNPYNDVPKNNAKFRG
CDR-H3:
(SEQ ID No: 22)
GGFFGPLSY
Light Chain Sequence (mouse Kappa isotype)
(SEQ ID No: 35)
DVLMTQTPLSLSVSLGDQASISCRSSQNIVYSNGDTYFEWYLQKPGQ
SPKLLIYKVSQRFSGVPDRFSGSGSGTDFTLKISRVETEDLGVYYCF
QGAHDPPAFGGGTKLELKRADAAPTVSIFPPSSEQLTSGGASVVCFL
NNFYPKDINVKWKIDGSERQNGVLNSWTDQDSKDSTYSMSSTLTLTK
DEYERHNSYTCEATHKTSTSPIVKSFNRNEC
CDR-L1:
(SEQ ID No: 17)
RSSQNIVYSNGDTYFE
CDR-L2:
(SEQ ID No: 18)
KVSQRFS
CDR-L3:
(SEQ ID No: 19)
FQGAHDPPA

### Example 3—Antibody Development for C4M

A monoclonal antibody specific for C4M was developed as previously described in Sand et. al.³⁸ (incorporated herein by reference) using the N-terminal neo-epitope sequence 162′-ILGHVPGMLL-′171 (SEQ ID No: 27) generated by MMP-12 cleavage between amino acids 161 and 162 of the α1 chain of type IV collagen as an immunogenic peptide. Briefly, generation of monoclonal antibodies was initiated by immunization of four to six-week-old Balb/C mice subcutaneously with 200 μl emulsified antigen and 50 μg of the immunogenic peptide (ILGHVPGMLL-GGC-KLH (SEQ ID No: 36)) using Freund's incomplete adjuvant. Immunizations were performed every 2nd week until stable sera titer levels were reached. The mouse with highest serum titer was selected for fusion. The mouse was rested for one month and then boosted intravenously with 50 μg of immunogenic peptide in 100 μl 0.9% sodium chloride solution three days before isolation of the spleen for cell fusion. Mouse spleen cells were fused with SP2/0 myeloma fusion partner cells. The resulting hybridoma cells were cloned using a semi-solid medium method, transferred into 96-well microtiter plates for further growth and incubated in a CO2 incubator. Standard limited dilution was used to promote monoclonal growth.

Native reactivity and peptide affinity of the monoclonal antibodies were evaluated by displacement of native samples (human, rat, and mouse serum, plasma, and urine) in a preliminary indirect ELISA using a biotinylated peptide (ILGHVPGMLL-K-biotin (SEQ ID No: 37)) on streptavidin-coated microtiter plates and the supernatant from the growing monoclonal hybridoma. Specificities of the clones to the free peptide (ILGHVPGMLL (SEQ ID No: 27)), a nonsense peptide, and an elongated peptide (EILGHVPGMLL (SEQ ID No: 28)) were tested. Isotyping of the monoclonal antibodies was performed using a SBA Clonotyping System-HRP kit. The monoclonal antibody was purified from collected supernatant of the selected clones using HiTrap protein G columns and subsequently labeled with horseradish peroxidase (HRP) using the Lightning link HRP labeling kit, according to the manufacturer's instructions.

The monoclonal antibody with the best native reactivity, peptide affinity, and stability was chosen from the antibody-producing clones generated after fusion between mouse spleen cells and myeloma cells. The clones selected were of the IgG1 subtype and the antibodies showed reactivity to healthy human, rat, and mouse serum, as well as human plasma EDTA, and showed no reactivity to the elongated peptide or nonsense peptide.

### Example 4—PRO-C3 Immunoassay

PRO-C3 was measured using an enzyme-linked immunosorbent assay (ELISA) developed at Nordic Bioscience, as described in WO2014/170312, and as also detailed in other publications¹⁹. Briefly, these procedures were as follows:

A 96-well streptavidin-coated ELISA plate from Roche, cat.11940279, was coated with the biotinylated peptide Biotin-CGG-CPTGPQNYSP (SEQ ID No: 33) dissolved in coater buffer (50 mM PBS-BTE+10% sorbitol, pH 7.4), incubated for 30 min at 20° C. in the dark and subsequently washed in washing buffer (20 mM Tris, 50 mM NaCl, pH 7.2). Thereafter 20 μl of peptide calibrator or sample were added to appropriate wells, followed by 100 μl of HRP-conjugated monoclonal antibody NB61N-62 dissolved in incubation buffer (50 mM PBS-BTB+10% LiquidII (Roche), pH 7.4) and the plate was incubated for 20 hours at 4° C. and washed. Finally, 100 μl tetramethylbenzinidine (TMB) (Kem-En-Tec cat.: 4380H) was added, the plate was incubated for 15 min at 20° C. in the dark and in order to stop the reaction, 100 μl of stopping solution (1% H₂SO₄) was added and the plate was analyzed in the ELISA reader at 450 nm with 650 nm as the reference (Molecular Devices, SpectraMax M, CA, USA). A calibration curve was plotted using a 4-parametric mathematical fit model.

### Example 5—PRO-C6 Immunoassay

PRO-C6 was measured using an enzyme-linked immunosorbent assay (ELISA) developed at Nordic Bioscience, as described in WO2016/156526, and as also detailed in other publications³⁹. Briefly, these procedures were as follows:

ELISA-plates used for the assay development were Streptavidin-coated from Roche (cat.: 11940279). All ELISA plates were analyzed with the ELISA reader from Molecular Devices, SpectraMax M, (CA, USA). We labeled the selected monoclonal antibody with horseradish peroxidase (HRP) using the Lightning link HRP labeling kit according to the instructions of the manufacturer (Innovabioscience, Babraham, Cambridge, UK). A 96-well streptavidin plate was coated with biotinylated synthetic peptide biotin-KPGVISVMGT (SEQ ID No: 38) (Chinese Peptide Company, China) dissolved in coating buffer (40 mM Na₂HPO₄, 7 mM KH₂PO₄, 137 mM NaCl, 2.7 mM KCl, 0.1% Tween 20, 1% BSA, pH 7.4) and incubated 30 minutes at 20° C. 20 μL of standard peptide or samples diluted in incubation buffer (40 mM Na₂HPO₄, 7 mM KH₂PO₄, 137 mM NaCl, 2.7 mM KCl, 0.1% Tween 20, 1% BSA, 5% Liquid II, pH 7.4) were added to appropriate wells, followed by 100 μL of HRP conjugated monoclonal antibody 10A3, and incubated 21 hour at 4° C. Finally, 100 μL tetramethylbenzinidine (TMB) (Kem-En-Tec cat.4380H) was added and the plate was incubated 15 minutes at 20° C. in the dark. All the above incubation steps included shaking at 300 rpm. After each incubation step the plate was washed five times in washing buffer (20 mM Tris, 50 mM NaCl). The TMB reaction was stopped by adding 100 μL of stopping solution (1% H₂SO₄) and measured at 450 nm with 650 nm as the reference.

### Example 6—C4M Immunoassay

C4M was measured using an enzyme-linked immunosorbent assay (ELISA) developed at Nordic Bioscience, as described in Sand et al³⁸. Briefly, these procedures were as follows:

A 96-well streptavidin-coated microtiter plate (cat. no. 11940279, Roche Diagnostics, Hvidovre, Denmark) was coated with 100 μl biotinylated peptide (ILGHVPGMLL-K-biotin (SEQ ID No: 37)) dissolved in coating buffer (50 mM Tris, containing 1% bovine serum albumin, 0.1% Tween-20, and 0.4% bronidox (BTB), pH 8.0) and incubated for 30 minutes at 20° C. 20 μl standard peptide or sample dissolved in assay buffer (50 mM Tris-BTB, pH 8.0) was added to appropriate wells, followed by 100 μl of conjugated monoclonal antibody diluted in assay buffer and incubated 1 hour at 20° C. Finally, 100 μl tetramethylbenzinidine (TMB) (cat. no. 4380H, Kem-En-Tec, Taastrup, Denmark) was added and the plate was incubated 15 minutes at 20° C. The TMB reaction was stopped by adding 100 μl stopping solution (1% H2SO4). All incubation steps were performed in the dark with shaking at 300 rpm and followed by five washes in washing buffer (20 mM Tris, 50 mM NaCl, pH 7.2). The results were analysed spectrophotometrically at 450 nm with 650 nm as the reference using an ELISA microplate reader (VersaMax, Molecular Devices, Sunnyvale, Calif., USA). A standard curve was performed by serial dilution of the standard peptide and plotted using a 4-parametric mathematical fit model.

### Example 7—Biomarker Analysis in TOPCAT Samples

In this study, the relationship between neoepitope biomarkers of type III, IV and VI collagen formation (Pro-C3, Pro-C4 and Pro-C6 respectively) and degradation (C3M, C4M and C6M, respectively) and subject outcomes, among subjects with HFpEF enrolled in the TOPCAT trial, was assessed.

Methods

Study Population

The study used data and biosamples from the TOPCAT Trial obtained from the National Heart, Lung, and Blood Institute. The parent trial data are available to other researchers through the National Institutes of Health Biolincc website.

The design of the TOPCAT trial and the general characteristics of the study population have been described in previous publications⁴⁶⁻⁴². Briefly, TOPCAT was a multi-center, international, randomized, double-blind, placebo-controlled trial of spironolactone that enrolled 3445 adults with HFpEF across >270 clinical sites in 6 countries from August 2006 until January 2012. The primary results of the trial have been previously published⁴². All study participants provided written informed consent.

Inclusion criteria for TOPCAT were as follows: age 50 years; diagnosis of HF based on at least 1 HF symptom at the time of study screening and at least 1 HF sign within the 12 months before screening; left ventricular EF≥45% (per local reading); at least 1 HF hospitalization in the 12 months before study screening or BNP (B-type natriuretic peptide) >100 pg/mL or NT-proBNP (N-terminal pro-BNP) >360 pg/mL (in the absence of an alternative explanation for elevated natriuretic peptide level) within the 60 days before screening; and serum potassium <5.0 mmol/L before randomization^{46,42}.

Exclusion criteria have been published in detail previously⁴⁰ but included severe systemic illness with a life expectancy of <3 years, significant chronic pulmonary disease, infiltrative or hypertrophic cardiomyopathy, constrictive pericarditis, previous cardiac transplant or LV assist device, known chronic hepatic disease, severe chronic kidney disease (defined as estimated glomerular filtration rate [eGFR]<30 mL/min per 1.73 m² or serum creatinine ≥2.5 mg/dL), a history of significant hyperkalemia, known intolerance to aldosterone antagonists, and recent myocardial infarction, coronary artery bypass grafting, or percutaneous coronary intervention.

The primary goal of the trial was to determine if spironolactone was associated with a reduction in the composite outcome of cardiovascular mortality, aborted cardiac arrest, or heart failure hospitalization. All HF hospitalizations were adjudicated by a clinical end point committee at Brigham and Women's Hospital, blinded to study-drug assignments, according to prespecified criteria, as previously described⁴⁰. In this analysis, we examined the relationship between biomarkers and tissue fibrosis and: (1) The primary endpoint, as defined above; (2) A composite endpoint of death or heart failure hospitalization, which is increasingly utilized in HFpEF studies⁴³.

Given significant regional variations in the trial population⁶, the analysis in the present study was limited to subjects enrolled in the Americas.

Biomarker Assays

Stored plasma samples were obtained from Biolincc for all participants enrolled in the Americas who had stored plasma from the baseline examination (n=206).

Specific biomarkers of collagen formation (Pro-C3, Pro-C4 and Pro-C6) and degradation (C3M, C4M and C6M) were measured using enzyme-linked immunosorbent assays (ELISA). The Pro-C3, Pro-C6 and C4M ELISAs were carried out as described supra (see Examples 4, 5 and 6, respectively). Pro-C4 is a known biomarker of collagen type IV formation, and the Pro-C4 ELISA was carried out in the manner described in Leeming et al⁴⁴. C3M and C6M are known biomarkers of collagen type III degradation and collagen type VI degradation, respectively, and the C3M and C6M ELISAs were carried out in the manner described in Barascuk et al⁴⁵ and Juhl et al⁴⁶, respectively.

NT-proBNP levels were measured using a validated Luminex Bead-Based multiplexed assay (Bristol Myers-Squibb; Ewing Township, N.J.).

Statistical Analyses

Participant characteristics were summarized using mean (SD) for normally distributed variables and median (interquartile range) for non-normally distributed continuous variables. Categorical variables are expressed as counts (percentages). Subjects enrolled in the Americas who had available samples for measurement of the biomarkers of interest vs. those who did not were compared. The non-paired t test for normally-distributed variables, the Kruskal-Wallis test for non-normally distributed variables and the chi-square test or Fisher's exact test, as appropriate, for categorical variables were used.

The relationship between biomarkers and the primary outcome (cardiovascular death, aborted cardiac arrest, or heart failure hospitalization), as well as the composite of HF hospitalization or all-cause death, were assessed using Cox regression. Kaplan-Meier survival curves for tertiles of each biomarker were constructed, and these were compared using the log-rank test. Adjusted Cox models were built, as appropriate, to assess whether unadjusted associations are independent of confounders, including: (1) the MAGGIC risk score, which incorporates multiple demographic, clinical and laboratory variables (Model 1)⁴⁷; (2) The MAGGIC risk score plus NT-proBNP levels (Model 2); (3) Important individual clinical covariates chosen a priori, including age, sex, diabetes mellitus status, estimated glomerular filtration rate, systolic blood pressure (SBP), and NYHA class III/IV and history of myocardial infarction (Model 3). Hazard ratios for all biomarkers are standardized (expressed per standard-deviation increase, or 1-point increased in the z score) in order to provide an intuitive comparison between the biomarkers.

Finally, interactions between the pre-randomization level of each biomarker and randomized treatment with spironolactone were tested, as predictors of the endpoints mentioned above. When an interaction was found, stratified survival analyses were performed according to the median value of the biomarker, in which the effect of spironolactone treatment was assessed.

Statistical significance was defined as a 2-tailed P value<0.05. All probability values presented are 2-tailed. Statistical analyses were performed using the Matlab statistics and machine learning toolbox (Matlab 2016b, the Mathworks; Natwick, Mass.) and SPSS for Mac v22 (SPSS Inc., Chicago, Ill.).

Results

A comparison of trial participants enrolled in the Americas who did vs. those who did not have available frozen biosamples for biomarker measurements is shown in Table 1. There were no significant differences between the subgroups in age or sex. Subjects with available samples demonstrated a slightly greater proportion of white (85.44 vs. 77.36%) and a slightly lower proportion of black (12.62 vs. 17.7%) participants. The prevalence of NYHA class history of myocardial infarction, stroke, peripheral arterial disease or diabetes mellitus did not differ between the subgroups, whereas the prevalence of COPD was lower and the prevalence of hypertension and atrial fibrillation was greater among participants with available samples. Antihypertensive medication, diuretic, glucose-lowering agent and ACE inhibitor/ARB use did not defer between the groups. Subjects with available samples were more often receiving stains.

TABLE 1
General characteristics of study participants with vs.
without available plasma samples. Numbers represent
Mean (SD), Median (IQR) or counts (%)

| | Participants | | Participants | | |
|---|---|---|---|---|---|
| | without | | with | | |
| | available | | available | | |
| | samples | | samples | | P |
| | (n = 1559) | | (n = 206) | | value |
| Demographic | | | | | |
| Characteristics | | | | | |
| Age, years | 72 | (64, 79) | 72 | (64, 79) | 0.9054 |
| Male Sex | 770 | (49.39%) | 113 | (54.85%) | 0.1405 |
| Race | | | | | <0.0001 |
| White | 1206 | (77.36%) | 176 | (85.44%) | 0.0082 |
| Black | 276 | (17.70%) | 26 | (12.62%) | 0.0687 |
| Asian | 18 | (1.15%) | 1 | (0.49%) | 0.7162* |
| Other | 67 | (4.30%) | 3 | (1.46%) | 0.0495 |
| BMI, kg/m2 | 32.8 | (27.9, 38.5) | 33.1 | (28.5, 37.8) | 0.5387 |
| Heart rate, bpm | 68 | (61, 76) | 66.5 | (60, 76) | 0.0456 |
| Systolic BP, mmHg | 130 | (118, 139) | 124 | (114, 136) | 0.0039 |
| Diastolic PB, mmHg | 70 | (62, 80) | 70 | (62, 78) | 0.0428 |
| Medical History | | | | | |
| NYHA class III-IV | 540 | (34.70%) | 80 | (38.83%) | 0.2434 |
| Myocardial | 313 | (20.09%) | 46 | (22.33%) | 0.4529 |
| Infarction | | | | | |
| Stroke | 143 | (9.18%) | 15 | (7.28%) | 0.3703 |
| COPD | 269 | (17.27%) | 22 | (10.68%) | 0.0167 |
| Hypertension | 1392 | (89.35%) | 195 | (94.66%) | 0.0170 |
| Peripheral Arterial | 183 | (11.75%) | 24 | (11.65%) | 0.9681 |
| Disease | | | | | |
| Atrial Fibrillation | 640 | (41.08%) | 102 | (49.51%) | 0.0212 |
| Diabetes Mellitus | 692 | (44.42%) | 96 | (46.60%) | 0.5531 |
| Medication Use | | | | | |
| Beta Blockers | 1215 | (77.98%) | 172 | (83.50%) | 0.0698 |
| Calcium Channel | 600 | (38.51%) | 81 | (39.32%) | 0.8225 |
| Blockers | | | | | |
| Diuretics | 1385 | (88.90%) | 187 | (90.78%) | 0.4153 |
| Glucose-lowering | 628 | (40.31%) | 91 | (44.17%) | 0.2885 |
| agents | | | | | |
| ACE Inhibitors or | 1240 | (79.59%) | 154 | (74.76%) | 0.1094 |
| ARBs | | | | | |
| Statins | 995 | (63.86%) | 153 | (74.27%) | 0.0032 |

Relationship Between Baseline Biomarkers of Tissue Fibrosis and Outcomes

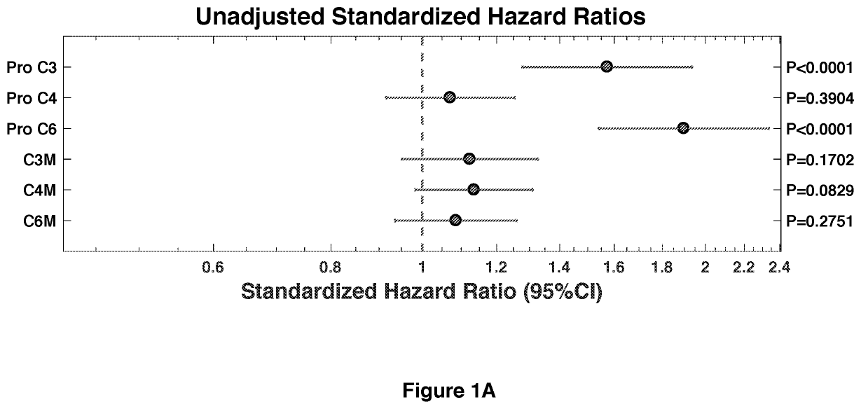

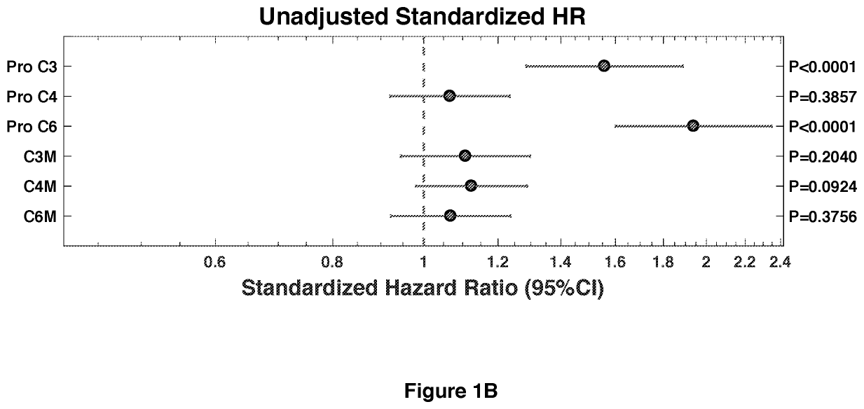

FIG. 1A shows standardized hazard ratios for all examined fibrosis biomarkers for the primary endpoint in unadjusted analyses (one model per biomarker). FIG. 1B shows corresponding standardized hazard ratios for death or heart failure admission. In these analyses, pro-C6 (HR=1.90; 95% CI=1.54-2.34; P<0.0001) and pro-C3 (HR=1.57; 95% CI=1.28-1.94; P<0.0001) strongly predicted the trial primary endpoint. Similarly, pro-C6 (HR=1.94; 95% CI=1.60-2.35; P<0.0001) and pro-C3 (HR=1.56; 95% CI=1.29-1.89; P<0.0001) predicted the composite endpoint of death or heart failure admission.

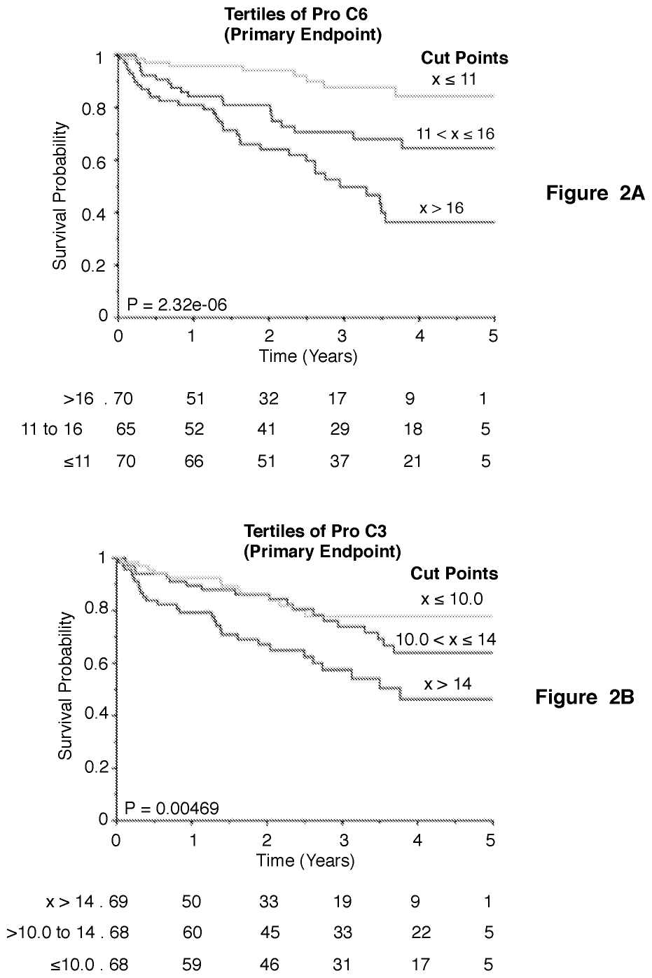

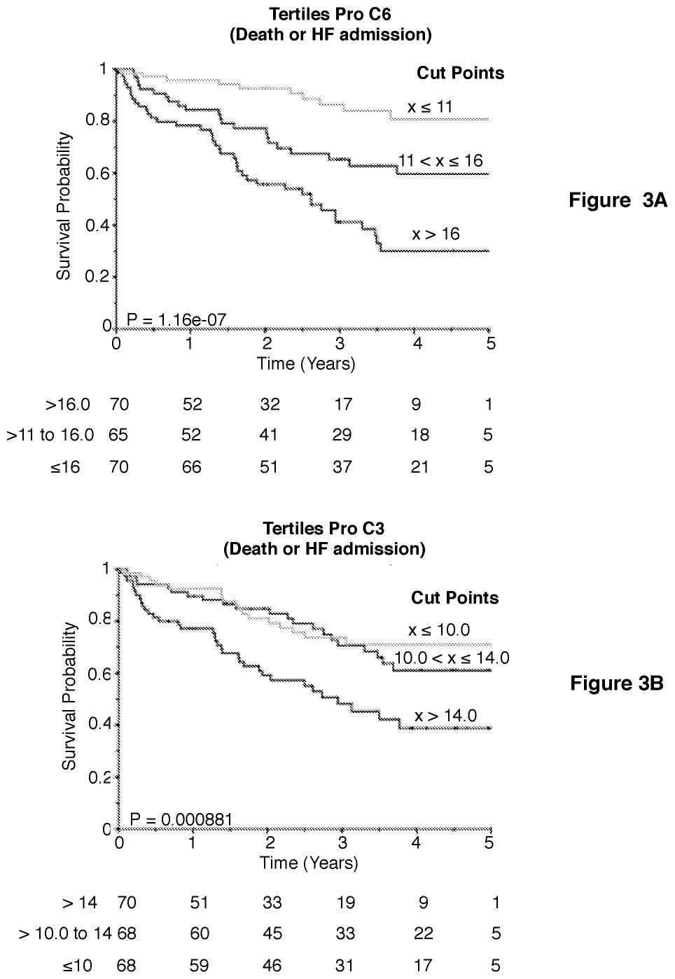

FIG. 2 shows Kaplan-Meier survival curves for the primary endpoint corresponding to the tertiles of Pro-C6 (left) and Pro-C3 (right), respectively. Pro-C6 stratified subjects across a broad range of absolute risk. There was graded pronounced reduction in event-free survival from the lowest tertile (Pro-C6<11.0 ng/ml) to the highest tertile (Pro-C6>16.0 ng/ml) of Pro-C6. For Pro-C3, only the highest tertile (Pro-C3>14.0 ng/ml) demonstrated a pronounced reduced event-free survival. A similar pattern was found for death or heart failure admission, as shown in FIG. 3.

In models that included both Pro-C6 and Pro-C3, pro-C6 was independently predictive of the primary endpoint (HR=1.84; 95% CI=1.36-2.47; P<0.0001) and of death/HF admission (HR=1.92; 95% CI=1.46-2.53; P<0.0001). In contrast, in these models, Pro-C3 was not significantly associated with either the primary endpoint (HR=1.06; 95% CI=0.76-1.46; P=0.74) or death/HF admission (HR=1.01; 95% CI=0.75-1.37; P=0.93). Similarly, in models that included both pro-C6 (as a continuous variable) and a pro-C3 level>14 ng/mL (highest tertile of distribution, expressed as a binary variable), pro-C6, but not pro-C3 status was independently predictive of the primary endpoint and of death/HF admission.

Subsequent adjusted analyses were performed for pro-C6 only (Table 2). In models that adjusted for the MAGGIC risk score, ProC6 strongly predict the primary endpoint (HR=1.88; 95% CI=1.52-2.33; P<0.0001) and death/HF admission (HR=1.91; 95% CI=1.57-2.33; P<0.0001). The hazard ratios for Pro-C6 were very similar when additional adjustment for NT-proBNP was performed (adjusted Model 2, Table 2). When adjusted for Pro-C6, NT-ProBNP became only weakly predictive of the outcome, whereas the MAGGIC risk score became non-predictive of the primary endpoint or of death/HF admission.

Similarly, in models that adjusted for age, sex, diabetes mellitus status, estimated glomerular filtration rate, SBP, NYHA class III/IV and history of myocardial infarction (adjusted Model 3, Table 2), ProC6 strongly predicted the primary endpoint (HR=1.81; 95% CI=1.44-2.27; P<0.0001) and the endpoint of death or HF admission (HR=1.84; 95% CI=1.49-2.26; P<0.0001).

As shown in Table 2, for the primary endpoint, the Harrel's C-statistic was much greater for a model including only pro-C6 alone (0.705) than for models including the MAGGIC risk score (0.552), the MAGGIC risk score plus BNP (0.582), or a combination of clinical variables included in adjusted model 3 (0.64). Similarly, for the death/heart failure-related hospitalization, the Harrel's C-statistic was much greater for a model including only pro-C6 alone (0.707) than for models including the MAGGIC risk score (Adjusted model 1: 0. 0.571), the MAGGIC risk score plus BNP (Adjusted model 2: 0.602), or a combination of clinical variables (Adjusted model 3: 0.623). Accordingly, the addition of pro-C6 to models already containing the MAGGIC risk score, the MAGGIC risk score plus BNP, or a combination of clinical variables, resulted in marked improvements in the Harrel's C-statistic (Table 2).

TABLE 2
Relationship between pro-C6 levels and the incidence of the primary
endpoint and of death or HF admission in various models.

| | | | Harrel's c | Harrel's c |
|---|---|---|---|---|
| | | | (with | (without |
| Model | HR (95% CI) | P value | ProC6)* | ProC6)* |
| Primary Endpoint | | | | |
| Unadjusted | 1.90 (1.54-2.34) | <0.0001 | — | 0.705 (0.034) |
| Adjusted Model 1 | 1.88 (1.52-2.33) | <0.0001 | 0.552 (0.041) | 0.699 (0.036) |
| Adjusted Model 2 | 1.87 (1.51-2.33) | <0.0001 | 0.552 (0.043) | 0.706 (0.035) |
| Adjusted Model 3 | 1.81 (1.44-2.27) | <0.0001 | 0.64 (0.042) | 0.724 (0.031) |
| Death or | | | | |
| HF admission | | | | |
| Unadjusted | 1.94 (1.60-2.35) | <0.0001 | — | 0.707 (0.031) |
| Adjusted Model 1 | 1.91 (1.57-2.33) | <0.0001 | 0.571 (0.037) | 0.698 (0.033) |
| Adjusted Model 2 | 1.90 (1.55-2.32) | <0.0001 | 0.602 (0.039) | 0.709 (0.032) |
| Adjusted Model 3 | 1.84 (1.49-2.26) | <0.0001 | 0.623 (0.039) | 0.715 (0.03) |

*The number in parentheses represents the standard error of the estimate.
Adjusted Model 1: adjusted for the MAGGIC risk score.
Adjusted Model 2: adjusted for the MAGGIC risk score and NT-proBNP levels.
Adjusted Model 3: adjusted for age, sex, diabetes mellitus, estimated glomerular filtration rate, systolic blood pressure (SBP), NYHA class III/IV and history of myocardial infarction.

Interactions with Randomized Arm

Significant interactions between baseline levels of C4M and randomized treatment arm were found as predictors of the primary endpoint (P for C4M-treatment arm interaction=0.0061) and of death/HF admission (P for C4M-treatment arm interaction=0.0063), indicating a more favorable response to treatment among participants with lower C4M levels at baseline. No interactions with treatment arm were found for the other examined fibrosis biomarkers.

Discussion

The relationship between biomarkers of ECM turnover, measured at baseline among participants enrolled in the TOPCAT trial was studied. It was demonstrated that pro-C6 and pro-C3, biomarkers of fibrogenesis assessed by type VI and III collagen formation, respectively, predicted the risk of incident cardiovascular events, as well as a composite of all-cause death/HF-related hospitalization in this population. Pro-C6, in particular, was a strong independent predictor of these outcomes and stratified subjects across a broad range of absolute risk. Pro-C6 alone performed better as a predictor of outcomes than the MAGGIC risk score, NT-ProBNP or a combination of clinical variables. The addition of pro-C6 to the MAGGIC risk score, with or without additional adjustment for NT-proBNP levels, resulted in a marked increase in the Harrel's C-statistic, a measure of model fit and discrimination which is analogous to the receiver-operator characteristic curve. In addition, an interaction between levels of C4M, a biomarker of collagen type IV degradation (present predominantly in the vascular basement membrane), and the risk reduction associated with randomization to spironolactone vs. placebo was found. In these post-hoc analyses, subjects with higher C4M levels appeared to derive greater benefit from randomization to spironolactone. These findings support an important role for tissue fibrosis in HFpEF, and identify biomarkers of ECM turnover that are readily measured, and could be implemented in various settings for risk stratification in this population.

In the present study, high levels of pro-C6 were strongly predictive of outcomes. It is important to note the pronounced prognostic power of this biomarker, which largely exceeded that of the MAGGIC risk score, the MAGGIC risk score plus NT-proBNP levels, and a combination of key clinical variables. In addition, pro-C6 markedly improved the discrimination of models that already included these prognostic factors; in contrast, the addition of standard predictors to a model already containing pro-C6 resulted in minimal improvements in model fit. Therefore, pro-C6 appears to be a particularly strong and robust independent predictor of outcomes in HFpEF. Pro-C6 may thus be useful in the diagnosis of HFpEF, for the identification of good candidates for antifibrotic therapies, and/or for monitoring and characterizing the efficacy of such therapies.

A particularly interesting finding of the present study is the highly significant interaction between C4M and the risk modification associated with randomized treatment with spironolactone. These findings support the notion that biomarkers of collagen turnover can identify individuals who benefit from spironolactone. The present study is the first that reports an interaction between biomarkers of collagen turnover and the reduction in the risk of clinical events associated with spironolactone therapy. It was found that lower C4M levels were associated with a greater reduction in risk associated with spironolactone randomization. C4M is a marker of collagen degradation; therefore, lower levels indicate reduced degradation and thus increased collagen accumulation, which is a therapeutic target of spironolactone.

In addition to blood vessels, collagen IV is also present in the glomerular basement membrane that prevents the leakage of plasma proteins into the urine. Interestingly, however, C4M was not associated with albuminuria in the present cohort. Similarly, in contrast to the pronounced interaction between changes in C4M and spironolactone, no interaction was found between proteinuria and spironolactone treatment in a recent analysis of the TOPCAT trial⁴⁸. Therefore, the interaction between C4M and spironolactone effects are unlikely to be mediated by glomerular basement membrane degradation.

In summary, fibrogenesis assessed by Pro-C6 is strongly and independently predictive of a poor prognosis in HFpEF. In contrast, low levels of C4M appear to identify patients with HFpEF who exhibit particularly favorable responses to aldosterone antagonists (mineralocorticoid-receptor antagonists).

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Lam C S, Donal E, Kraigher-Krainer E, Vasan R S. Epidemiology and clinical course of heart failure with preserved ejection fraction. Eur J Heart Fail 2011; 13:18-28.
2. Lloyd-Jones D M, Hong Y, Labarthe D et al. Defining and setting national goals for cardiovascular health promotion and disease reduction: the American Heart Association's strategic Impact Goal through 2020 and beyond. Circulation 2010; 121:586-613.
3. Lam C S, Donal E, Kraigher-Krainer E, Vasan R S. Epidemiology and clinical course of heart failure with preserved ejection fraction. Eur J Heart Fail 2011; 13:18-28.
4. Chirinos J A. Deep Phenotyping of Systemic Arterial Hemodynamics in HFpEF (Part 2): Clinical and Therapeutic Considerations. J Cardiovasc Transl Res 2017; 10:261-274.
5. Rommel K P, von Roeder M, Latuscynski K et al. Extracellular Volume Fraction for Characterization of Patients With Heart Failure and Preserved Ejection Fraction. J Am Coll Cardiol 2016; 67:1815-25.
6. Mohammed S F, Hussain S, Mirzoyev S A, Edwards W D, Maleszewski J J, Redfield M M. Coronary microvascular rarefaction and myocardial fibrosis in heart failure with preserved ejection fraction. Circulation 2015; 131:550-9.
7. Chirinos J A, Akers S R, Trieu L et al. Heart Failure, Left Ventricular Remodeling, and Circulating Nitric Oxide Metabolites. J Am Heart Assoc 2016; 5.
8. Su M Y, Lin L Y, Tseng Y H et al. CMR-verified diffuse myocardial fibrosis is associated with diastolic dysfunction in HFpEF. JACC Cardiovasc Imaging 2014; 7:991-7.
9. Mohammed S F, Majure D T, Redfield M M. Zooming in on the Microvasculature in Heart Failure With Preserved Ejection Fraction. Circ Heart Fail 2016; 9.
10. Richards A M. Circulating Biomarkers of Cardiac Fibrosis: Do We Have Any and What Use Are They? Circ Heart Fail 2017; 10.
11. Lin L Y, Wu C K, Juang J M et al. Myocardial Regional Interstitial Fibrosis is Associated With Left Intra-Ventricular Dyssynchrony in Patients With Heart Failure: A Cardiovascular Magnetic Resonance Study. Sci Rep 2016; 6:20711.
12. Duca F, Kammerlander A A, Zotter-Tufaro C et al. Interstitial Fibrosis, Functional Status, and Outcomes in Heart Failure With Preserved Ejection Fraction: Insights From a Prospective

Cardiac Magnetic Resonance Imaging Study. Circ Cardiovasc Imaging 2016; 9.
13. Roy C, Slimani A, de Meester C et al. Associations and prognostic significance of diffuse myocardial fibrosis by cardiovascular magnetic resonance in heart failure with preserved ejection fraction. J Cardiovasc Magn Reson 2018; 20:55.
14. Schelbert E B, Fridman Y, Wong T C et al. Temporal Relation Between Myocardial Fibrosis and Heart Failure With Preserved Ejection Fraction: Association With Baseline Disease Severity and Subsequent Outcome. JAMA Cardiol 2017.
15. Nielsen M J, Karsdal M A, Kazankov K et al. Fibrosis is not just fibrosis-basement membrane modelling and collagen metabolism differs between hepatitis B- and C-induced injury. Aliment Pharmacol Ther 2016; 44:1242-1252.
16. Haykowsky M J, Kouba E J, Brubaker P H, Nicklas B J, Eggebeen J, Kitzman D W. Skeletal muscle composition and its relation to exercise intolerance in older patients with heart failure and preserved ejection fraction. Am J Cardiol 2014; 113:1211-6.
17. Rossignol P, Ferreira J P, Zannad F. Fibrosis mechanistic phenotyping and antifibrotic response determination with biomarkers in heart failure: one single biomarker may not fit all settings. Eur J Heart Fail 2018; 20:1300-1302.
18. Ravassa S, Trippel T, Bach D et al. Biomarker-based phenotyping of myocardial fibrosis identifies patients with heart failure with preserved ejection fraction resistant to the beneficial effects of spironolactone: results from the Aldo-DHF trial. Eur J Heart Fail 2018; 20:1290-1299.
19. Nielsen M J, Nedergaard A F, Sun S et al. The neo-epitope specific PRO-C3 ELISA measures true formation of type III collagen associated with liver and muscle parameters. Am. J. Transl. Res. 2013; 5: 303-315.
20. Nielsen M J, Veidal S S, Karsdal M A et al. Plasma Pro-C3 (N-terminal type III collagen propeptide) predicts fibrosis progression in patients with chronic hepatitis C. Liver Int. 2015; 35: 429-437.
21. Daniels S J, Leeming D J, Eslam M et al. ADAPT: An algorithm incorporating PRO-C3 accurately identifies patients with NAFLD and advanced fibrosis. Hepatology 2018.
22. Leeming D J, Karsdal M A, Byrjalsen I et al. Novel serological neo-epitope markers of extracellular matrix proteins for the detection of portal hypertension. Aliment. Pharmacol. Ther. 2013; 38: 1086-96.
23. Jansen C, Leeming D J, Mandorfer M et al. PRO-C3-Levels in Patients with HIV/HCV-Co-Infection Reflect Fibrosis Stage and Degree of Portal Hypertension. Avila M A, ed. PLoS One 2014; 9: e108544.
24. Karsdal M A, Hjuler S T, Luo Y et al. Assessment of liver fibrosis progression and regression by a serological collagen turnover profile. Am. J. Physiol. Gastrointest. Liver Physiol. 2019; 316: G25-G31.
25. Nielsen S H, Mygind N D, Michelsen M M et al. Accelerated collagen turnover in women with angina pectoris without obstructive coronary artery disease: An iPOWER substudy. Eur. J. Prev. Cardiol. 2018; 25: 719-727.
26. Hansen J F, Juul Nielsen M, Nyström K et al. PRO-C3: a new and more precise collagen marker for liver fibrosis in patients with chronic hepatitis C. Scand. J. Gastroenterol. 2018; 53: 83-87.
27. Park J, Scherer P E, Calle E et al. Adipocyte-derived endotrophin promotes malignant tumor progression. J. Clin. Invest. 2012; 122: 4243-4256.
28. Lee C, Kim M, Lee J H et al. COL6A3-derived endotrophin links reciprocal interactions among hepatic cells in the pathology of chronic liver disease. J. Pathol. 2018.
29. Sun K, Park J, Gupta O T et al. Endotrophin triggers adipose tissue fibrosis and metabolic dysfunction. Nat. Commun. 2014; 5: 3485.
30. Park J, Morley T S, Scherer P E. Inhibition of endotrophin, a cleavage product of collagen VI, confers cisplatin sensitivity to tumours. EMBO Mol. Med. 2013; 5: 935-48.
31. Bu D, Crewe C, Kusminski C M et al. Human endotrophin as a driver of malignant tumor growth. JCI Insight 2019.
32. Rasmussen D G K, Hansen T W, von Scholten B J et al. Higher Collagen VI Formation Is Associated With All-Cause Mortality in Patients With Type 2 Diabetes and Microalbuminuria. Diabetes Care 2018: dc172392.
33. Fenton A, Jesky M D, Ferro C J et al. Serum endotrophin, a type VI collagen cleavage product, is associated with increased mortality in chronic kidney disease. Aguilera A I, ed. PLoS One 2017; 12: e0175200.
34. Rasmussen D G K, Fenton A, Jesky M et al. Urinary endotrophin predicts disease progression in patients with chronic kidney disease. Sci. Rep. 2017; 7: 17328.
35. Karsdal M A, Henriksen K, Genovese F et al. Serum endotrophin identifies optimal responders to PPARγ agonists in type 2 diabetes. Diabetologia 2017; 60.
36. Timpl R. Macromolecular organization of basement membranes. Curr Opin Cell Biol 1996; 8:618-24.
37. Jayadev R, Sherwood D R. Basement membranes. Curr Biol 2017; 27:R207-R211.
38. Sand J M, Larsen L, Hogaboam C et al. MMP mediated degradation of type IV collagen alpha 1 and alpha 3 chains reflects basement membrane remodeling in experimental and clinical fibrosis—validation of two novel biomarker assays. PLoS One 2013; 8:e84934.
39. Sun S, Henriksen K, Karsdal M A et al. Collagen Type III and VI Turnover in Response to Long-Term Immobilization. PLoS One 2015; 10: e0144525.
40. Desai A S, Lewis E F, Li R et al. Rationale and design of the Treatment of Preserved Cardiac Function Heart Failure with an Aldosterone Antagonist Trial: A randomized, controlled study of spironolactone in patients with symptomatic heart failure and preserved ejection fraction. Am. Heart J. 2011; 162: 966-972.e10.
41. Pfeffer M A, Claggett B, Assmann S F et al. Regional variation in patients and outcomes in the Treatment of Preserved Cardiac Function Heart Failure With an Aldosterone Antagonist (TOPCAT) trial. Circulation 2015; 131:34-42.
42. Pitt B, Pfeffer M A, Assmann S F et al. Spironolactone for heart failure with preserved ejection fraction. N Engl J Med 2014; 370:1383-92.
43. Lam C S P, Gamble G D, Ling L H et al. Mortality associated with heart failure with preserved vs. reduced ejection fraction in a prospective international multi-ethnic cohort study. Eur Heart J 2018; 39:1770-1780.
44. Leeming D J, Nielsen M J, Dai Y et al. Enzyme-linked immunosorbent serum assay specific for the 7S domain of Collagen Type IV (P4NP 7S): A marker related to the extracellular matrix remodeling during liver fibrogenesis. Hepatol Res 2012; 42:482-93.
45. Barascuk N, Veidal S S, Larsen L et al. A novel assay for extracellular matrix remodeling associated with liver fibrosis: An enzyme-linked immunosorbent assay (ELISA) for a MMP-9 proteolytically revealed neo-epitope of type III collagen. Clin Biochem 2010; 43:899-904.
46. Juhl P, Bay-Jensen A C, Karsdal M, Siebuhr A S, Franchimont N, Chavez J. Serum biomarkers of collagen turnover as potential diagnostic tools in diffuse systemic sclerosis: A cross-sectional study. PLoS One 2018; 13:e0207324.
47. Pocock S J, Ariti C A, McMurray J J et al. Predicting survival in heart failure: a risk score based on 39 372 patients from 30 studies. Eur Heart J 2013; 34:1404-13.
48. Selvaraj S, Claggett B, Shah S J et al. Prognostic Value of Albuminuria and Influence of Spironolactone in Heart Failure With Preserved Ejection Fraction. Circ Heart Fail 2018; 11:e005288.

1: A method of immunoassay for detecting and/or monitoring a cardiovascular disease in a patient and/or assessing the likelihood of or the severity of a cardiovascular disease in a patient, wherein said method comprises:
(i) contacting a biofluid sample from a patient with a monoclonal antibody that specifically binds to a C-terminal epitope of the C5 domain of the α3 chain of type VI collagen, and/or contacting a biofluid sample from the patient with a monoclonal antibody that specifically binds to a C-terminal neo-epitope of the N-terminal propeptide of type III collagen,
(ii) detecting and determining the amount of binding between each monoclonal antibody used in step (i) and peptides in the sample or samples, and
(iii) correlating said amount of binding of each monoclonal antibody as determined in step (ii) with values associated with normal healthy subjects and/or values associated with known disease severity and/or values obtained from said patient at a previous time point and/or a predetermined cut-off value.

2: The method of claim 1, wherein the cardiovascular disease is heart failure.

3: The method of claim 2, wherein the cardiovascular disease is heart failure with a preserved ejection fraction (HFpEF).

4: The method of claim 1, wherein the method is a method for assessing the severity of a cardiovascular disease in a patient that comprises assessing the likelihood of patient mortality and/or hospitalization as a result of the cardiovascular disease and/or a composite of adverse cardiovascular events.

5: The method of claim 1, wherein the patient is a patient undergoing a therapy for the cardiovascular disease.

6: The method of claim 1, wherein step (i) comprises contacting a biofluid sample from the patient with a monoclonal antibody that specifically binds to a C-terminal epitope of the C5 domain of the α3 chain of type VI collagen.

7: The method of claim 6, wherein said monoclonal antibody specifically binds to a C-terminus amino acid sequence KPGVISVMGT (SEQ ID NO: 1).

8: The method of claim 7, wherein said monoclonal antibody does not recognize or specifically bind to an elongated version of said C-terminus amino acid sequence which is KPGVISVMGTA (SEQ ID NO: 2), or to a truncated version of said C-terminus amino acid sequence which is KPGVISVMG (SEQ ID NO: 3).

9: The method of claim 1, wherein step (i) comprises contacting a biofluid sample from the patient with a monoclonal antibody that specifically binds to a C-terminal neo-epitope of the N-terminal propeptide of type III collagen.

10: The method of claim 9, wherein said monoclonal antibody specifically binds to a C-terminus amino acid sequence CPTGPQNYSP (SEQ ID NO: 14).

11: The method of claim 10, wherein said monoclonal antibody does not recognize or specifically bind to an elongated version of said C-terminus amino acid sequence which is CPTGPQNYSPQ (SEQ ID NO: 15), or to a truncated version of said C-terminus amino acid sequence which is CPTGPQNYS (SEQ ID NO: 16).

12: The method of claim 1, wherein said biofluid is serum or plasma.

13: The method claim 1, wherein said immunoassay is a competition assay or a sandwich assay.

14: The method of claim 1, wherein said immunoassay is a radioimmunoassay or an enzyme-linked immunosorbent assay.

15: A method of monitoring cardiovascular disease and/or assessing the severity of a cardiovascular disease in a patient undergoing treatment with an aldosterone antagonist, wherein said method comprises:
(i) contacting a biofluid sample from a patient undergoing treatment with an aldosterone antagonist with a monoclonal antibody that specifically binds to a C-terminal epitope of the C5 domain of the α3 chain of type VI collagen, and/or contacting a biofluid sample from the patient with a monoclonal antibody that specifically binds to a C-terminal neo-epitope of the N-terminal propeptide of type III collagen,
(ii) detecting and determining the amount of binding between each monoclonal antibody used in step (i) and peptides in the sample or samples, and
(iii) correlating said amount of binding of each monoclonal antibody as determined in step (ii) with values associated with normal healthy subjects and/or values associated with known disease severity and/or values obtained from said patient at a previous time point and/or a predetermined cut-off value.

16: The method of claim 15, wherein the aldosterone antagonist is Spironolactone.

17: The method of claim 15, wherein the cardiovascular disease is heart failure.

18: The method of claim 17, wherein the cardiovascular disease is heart failure with a preserved ejection fraction (HFpEF).

19: The method of claim 15, wherein the method is a method for assessing the severity of a cardiovascular disease in a patient that comprises assessing the likelihood of patient mortality and/or hospitalization as a result of the cardiovascular disease and/or a composite of adverse cardiovascular events.

20: The method of claim 15, wherein step (i) comprises contacting a biofluid sample from the patient with a monoclonal antibody that specifically binds to a C-terminal epitope of the C5 domain of the α3 chain of type VI collagen.

21: The method of claim 20, wherein said monoclonal antibody specifically binds to a C-terminus amino acid sequence KPGVISVMGT (SEQ ID NO: 1).

22: The method of claim 21, wherein said monoclonal antibody does not recognize or specifically bind to an elongated version of said C-terminus amino acid sequence which is KPGVISVMGTA (SEQ ID NO: 2), or to a truncated version of said C-terminus amino acid sequence which is KPGVISVMG (SEQ ID NO: 3).

23: The method of claim 15, wherein step (i) comprises contacting a biofluid sample from the patient with a monoclonal antibody that specifically binds to a C-terminal neo-epitope of the N-terminal propeptide of type III collagen.

24: The method of claim 23, wherein said monoclonal antibody specifically binds to a C-terminus amino acid sequence CPTGPQNYSP (SEQ ID NO: 14).

25: The method of claim 24, wherein said monoclonal antibody does not recognize or specifically bind to an elongated version of said C-terminus amino acid sequence which is CPTGPQNYSPQ (SEQ ID NO: 15), or to a truncated version of said C-terminus amino acid sequence which is CPTGPQNYS (SEQ ID NO: 16).

26: The method of claim 15, wherein said biofluid is serum or plasma.

27: The method of claim 15, wherein said immunoassay is a competition assay or a sandwich assay.

28: The method of claim 15, wherein said immunoassay is a radioimmunoassay or an enzyme-linked immunosorbent assay.


SEQUENCE LISTING (38 sequences)

SEQ ID NO: 1 - protein, 10 aa, Artificial Sequence
  C-terminal epitope sequence PRO-C6 from type VI collagen alpha3 chain
KPGVISVMGT

SEQ ID NO: 2 - protein, 11 aa, Artificial Sequence
  Elongated PRO-C6 sequence
KPGVISVMGTA

SEQ ID NO: 3 - protein, 9 aa, Artificial Sequence
  Truncated PRO-C6 sequence
KPGVISVMG

SEQ ID NO: 4 - protein, 16 aa, Artificial Sequence
  PRO-C6 antibody complementarity-determining region CDR-L1
RSSQRIVHSNGITFLE

SEQ ID NO: 5 - protein, 7 aa, Artificial Sequence
  PRO-C6 antibody complementarity-determining region CDR-L2
RVSNRFS

SEQ ID NO: 6 - protein, 9 aa, Artificial Sequence
  PRO-C6 antibody complementarity-determining region CDR-L3
FQGSHVPLT

SEQ ID NO: 7 - protein, 5 aa, Artificial Sequence
  PRO-C6 antibody complementarity-determining region CDR-H1
DFNMN

SEQ ID NO: 8 - protein, 17 aa, Artificial Sequence
  PRO-C6 antibody complementarity-determining region CDR-H2
AINPHNGATSYNQKFSG

SEQ ID NO: 9 - protein, 7 aa, Artificial Sequence
  PRO-C6 antibody complementarity-determining region CDR-H3
WGNGKNS

SEQ ID NO: 10 - protein, 79 aa, Artificial Sequence
  PRO-C6 antibody light chain partial sequence
RSSQRIVHSNGITFLEWYLQKPGQSPKLLIYRVSNRFSGVPDRFSGSGSGTDFTLKISRVEAEDLGLYYCFQGSHVPLT

SEQ ID NO: 11 - protein, 75 aa, Artificial Sequence
  PRO-C6 antibody heavy chain partial sequence
DFNMNWVKQSHGKSLEWIGAINPHNGATSYNQKFSGKATLTVDKSSSTAYMELNSLTSDDSAVYYCARWGNGKNS

SEQ ID NO: 12 - protein, 112 aa, Artificial Sequence
  PRO-C6 antibody light chain variable region sequence
DVVMTQTPLSLPVNLGDQASISCRSSQRIVHSNGITFLEWYLQKPGQSPKLLIYRVSNRFSGVPDRFSGSGSGTDFTLKISRVEAEDLGLYYCFQGSHVPLTFGAGTRLELK

SEQ ID NO: 13 - protein, 116 aa, Artificial Sequence
  PRO-C6 antibody heavy chain variable region sequence
EVQLQQSGPVMVKPGTSVKTSCKASGYTFTDFNMNWVKQSHGKSLEWIGAINPHNGATSYNQKFSGKATLTVDKSSSTAYMELNSLTSDDSAVYYCARWGNGKNSWGQGTTLTVSS

SEQ ID NO: 14 - protein, 10 aa, Artificial Sequence
  PRO-C3 C-terminal neoepitope sequence from N-terminal propeptide of type III collagen (PIIINP)
CPTGPQNYSP

SEQ ID NO: 15 - protein, 11 aa, Artificial Sequence
  PRO-C3 elongated sequence
CPTGPQNYSPQ

SEQ ID NO: 16 - protein, 9 aa, Artificial Sequence
  PRO-C3 truncated sequence
CPTGPQNYS

SEQ ID NO: 17 - protein, 16 aa, Artificial Sequence
  PRO-C3 antibody complementarity-determining region CDR-L1
RSSQNIVYSNGDTYFE

SEQ ID NO: 18 - protein, 7 aa, Artificial Sequence
  PRO-C3 antibody complementarity-determining region CDR-L2
KVSQRFS

SEQ ID NO: 19 - protein, 9 aa, Artificial Sequence
  PRO-C3 antibody complementarity-determining region CDR-L3
FQGAHDPPA

SEQ ID NO: 20 - protein, 10 aa, Artificial Sequence
  PRO-C3 antibody complementarity-determining region CDR-H1
GYTFINYVIH

SEQ ID NO: 21 - protein, 17 aa, Artificial Sequence
  PRO-C3 antibody complementarity-determining region CDR-H2
YMNPYNDVPKNNAKFRG

SEQ ID NO: 22 - protein, 9 aa, Artificial Sequence
  PRO-C3 antibody complementarity-determining region CDR-H3
GGFFGPLSY

SEQ ID NO: 23 - protein, 79 aa, Artificial Sequence
  PRO-C3 antibody light chain partial sequence
RSSQNIVYSNGDTYFEWYLQKPGQSPKLLIYKVSQRFSGVPDRFSGSGSGTDFTLKISRVETEDLGVYYCFQGAHDPPA

SEQ ID NO: 24 - protein, 82 aa, Artificial Sequence
  PRO-C3 antibody heavy chain partial sequence
GYTFINYVIHWLKQKAGQGPEWIGYMNPYNDVPKNNAKFRGKARLTSDRSSTTAYMELNSLTSEDSAVYYCARGGFFGPLSY

SEQ ID NO: 25 - protein, 112 aa, Artificial Sequence
  PRO-C3 antibody light chain variable region sequence
DVLMTQTPLSLSVSLGDQASISCRSSQNIVYSNGDTYFEWYLQKPGQSPKLLIYKVSQRFSGVPDRFSGSGSGTDFTLKISRVETEDLGVYYCFQGAHDPPAFGGGTKLELK

SEQ ID NO: 26 - protein, 118 aa, Artificial Sequence
  PRO-C3 antibody heavy chain variable region sequence
EVQLQQSGPEVLKPGASVKMSCKASGYTFINYVIHWLKQKAGQGPEWIGYMNPYNDVPKNNAKFRGKARLTSDRSSTTAYMELNSLTSEDSAVYYCARGGFFGPLSYWGQGTLVTVSA

SEQ ID NO: 27 - protein, 10 aa, Artificial Sequence
  C4M N-terminal neoepitope sequence from cleavage of alpha1(IV) chain of type IV collagen
ILGHVPGMLL

SEQ ID NO: 28 - protein, 11 aa, Artificial Sequence
  C4M elongated sequence
EILGHVPGMLL

SEQ ID NO: 29 - protein, 9 aa, Artificial Sequence
  C4M truncated sequence
LGHVPGMLL

SEQ ID NO: 30 - protein, 440 aa, Artificial Sequence
  PRO-C6 antibody heavy chain sequence
EVQLQQSGPVMVKPGTSVKTSCKASGYTFTDFNMNWVKQSHGKSLEWIGAINPHNGATSYNQKFSGKATLTVDKSSSTAYMELNSLTSDDSAVYYCARWGNGKNSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 31 - protein, 219 aa, Artificial Sequence
  PRO-C6 antibody light chain sequence
DVVMTQTPLSLPVNLGDQASISCRSSQRIVHSNGITFLEWYLQKPGQSPKLLIYRVSNRFSGVPDRFSGSGSGTDFTLKISRVEAEDLGLYYCFQGSHVPLTFGAGTRLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 13 aa, Artificial Sequence
  PRO-C3 immunogenic peptide sequence
  SITE 1: Linked to ovalbumin
CGGCPTGPQNYSP

SEQ ID NO: 33 - protein, 13 aa, Artificial Sequence
  PRO-C3 screening peptide
  SITE 1: Linked to biotin
CGGCPTGPQNYSP

SEQ ID NO: 34 - protein, 224 aa, Artificial Sequence
  PRO-C3 antibody heavy chain sequence
EVQLQQSGPEVLKPGASVKMSCKASGYTFINYVIHWLKQKAGQGPEWIGYMNPYNDVPKNNAKFRGKARLTSDRSSTTAYMELNSLTSEDSAVYYCARGGFFGPLSYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKP

SEQ ID NO: 35 - protein, 219 aa, Artificial Sequence
  PRO-C3 antibody light chain sequence
DVLMTQTPLSLSVSLGDQASISCRSSQNIVYSNGDTYFEWYLQKPGQSPKLLIYKVSQRFSGVPDRFSGSGSGTDFTLKISRVETEDLGVYYCFQGAHDPPAFGGGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 36 - protein, 13 aa, Artificial Sequence
  C4M immunogenic peptide sequence
  SITE 13: linked to keyhole limpet hemocyanin
ILGHVPGMLLGGC

SEQ ID NO: 37 - protein, 11 aa, Artificial Sequence
  C4M biotinylated peptide sequence
  SITE 11: linked to biotin
ILGHVPGMLLK

SEQ ID NO: 38 - protein, 10 aa, Artificial Sequence
  PRO-C6 biotinylated peptide sequence
  SITE 1: Linked to biotin
KPGVISVMGT